(19) United States
(12) Patent Application Publication
(10) Pub. No.: US 2017/0020794 A1
(43) Pub. Date: Jan. 26, 2017

(54) AGENT CONTAINING MICROCAPSULES

(71) Applicant: Henkel AG & Co. KGaA, Duesseldorf (DE)

(72) Inventors: Andreas Bauer, Kaarst (DE); Ursula Huchel, Koeln (DE); Stefan Urlichs, Essen (DE); Manuela Materne, Kaarst (DE); Klaus Last, Braunschweig (DE); Daniel Mues, Hannover (DE)

(73) Assignee: Henkel AG & Co. KGaA, Duesseldorf (DE)

(21) Appl. No.: 15/288,584

(22) Filed: Oct. 7, 2016

Related U.S. Application Data
(62) Division of application No. 14/202,817, filed on Mar. 10, 2014, now abandoned.
Application No. 14/202,817 is a continuation of application No. PCT/EP2012/065640, filed on Aug. 10, 2012.

(30) Foreign Application Priority Data
Sep. 12, 2011 (DE) 10 2011 082 496.0

(51) Int. Cl. A61K 8/11; A61Q 19/00; A61Q 5/02; C11D 17/00; A61Q 5/10; C11D 3/37; C11D 3/50; A61Q 5/12; A61Q 19/10

(52) U.S. Cl. CPC A61K 8/11; A61Q 5/12; A61Q 19/00; A61Q 5/02; A61Q 19/10; A61Q 5/10; C11D 3/378; C11D 3/3703; C11D 3/505; C11D 17/0039

(57) ABSTRACT

The application relates to a method of using cleaning agents, and fabric treatment agents containing a combination of at least two different microcapsules as well as to methods for releasing active substances from said microcapsules when using said agents.

### CROSS-REFERENCE TO RELATED APPLICATION

This application is a divisional of U.S. application Ser. No. 14/202,817, filed on Mar. 10, 2014, which is a national phase entry of PCT Application Serial No. PCT/EP2012/065640, filed on Aug. 10, 2012, and entitled “AGENT CONTAINING MICROCAPSULES”, which claims priority to German Patent Application No. 10 2011 082 496.0, filed Sep. 12, 2011, each of which are hereby incorporated by reference in their entirety.

### FIELD OF THE INVENTION

The present invention generally relates to methods of using cleaning agents and fabric treatment agents which comprise microcapsules, as well as to methods for releasing active substances from these microcapsules when using said agents.

### BACKGROUND OF THE INVENTION

Many cosmetic agents, cleaning agents and fabric treatment agents comprise sensitive ingredients, such as e.g. fragrances or plant extracts. Disadvantageously, the types of ingredients that are incorporated in such agents frequently lose their activity during storage and/or their activity is at least strongly diminished before the desired time of application, namely for example by chemical reactions as a result of interactions with other constituents of the agent in question and/or due to physical factors. For this reason an encapsulation of certain ingredients is advantageous.

Numerous commercial encapsulation systems already exist which are based on naturally occurring or synthetic polymers. They can enclose an active substance or its solution and then be physically or chemically crosslinked in the shell or be precipitated out with another polymer by a coacervation process. Microcapsules that can comprise liquid, solid or gaseous substances as the core material are known from the prior art. Phenol-formaldehyde polymers, melamin-formaldehyde polymers, polyurethane, gelatin, polyamides or polyureas can be used as the material for the capsule walls.

Cosmetic agents, cleaning agents and fabric treatment agents which comprise microcapsules are known as such. Due to their particular stability, microcapsules, in particular made of melamin-formaldehyde resins, have proven their worth in these agents. There is, however, a problem, in that in the manufacture of these microcapsules, the obtained capsule dispersions basically still include residual free formaldehyde, the presence of which, in further processing or in the end product that is supplied to the consumer, is undesirable. Consequently, the patent literature contains proposals to lower the formaldehyde content by adding formaldehyde scavengers. Thus, different microcapsules and methods for their production are described in EP 0 383 358 and U.S. Pat. No. 4,918,317 with the aim of removing excess formaldehyde with the aid of formaldehyde scavengers.

The formaldehyde content of the dispersion is usually lowered by adding the cited formaldehyde scavengers to the microcapsule dispersion or during the manufacture of the microcapsule dispersion. However, the formaldehyde content of products that this type of microcapsule dispersions comprise or which were treated with them, often cannot be reduced below a certain level, even by adding large quantities of formaldehyde scavengers.

Therefore, it is of general interest to provide microcapsule-containing cosmetic compositions as well as cleaning agents or fabric treatment agents that comprise microcapsules, which involve the lowest possible amount of formaldehyde or in which the use of formaldehyde for microcapsules is preferably totally avoided.

Formaldehyde-free microcapsules are produced for example from a resin made of an aromatic alcohol and aldehydic components. Formaldehyde-free microcapsules are described in WO 2010/102830 A2. Some formaldehyde-free microcapsules produced from a resin made of an aromatic alcohol and aldehydic components bring about almost no color change in the end product and after longer storage times show only a weak sedimentation of the capsules, but however, to some extent, if they were loaded with an active substance, still exhibit only a weak boost effect (release of the active substance by friction) after protracted storage. Other formaldehyde-free microcapsules from a resin made of an aromatic alcohol and aldehydic components have again the advantage that even after protracted storage they still achieve a very strong boost effect, but poorer results in regard to sedimentation when stored, and exhibit color changes of the end product. Consequently there is still a need for formaldehyde-free microcapsules that possess good properties in regard to color fastness of the agent, storage stability and release of the active substance.

Accordingly, the object of the present invention was to develop formaldehyde-free microcapsules with good boost properties and good storage stability, and which do not lead to any impairment in the color of the end product.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An agent for washing, cleaning, conditioning, caring and/or dyeing hard or soft surfaces, comprising at least one surface-active substance, and a mixture of at least a first and a second microcapsule, each of whose capsule walls comprise a resin that is obtained by reacting a) at least one aromatic alcohol or its ethers or derivatives with b) at least one aldehydic component that possesses at least two carbon atoms per molecule, and c) optionally in the presence of at least one (meth)acrylate polymer, wherein the first and the second microcapsules differ from one another in at least one of the reacted components a) and/or b).

A method for releasing an active substance from a microcapsule that is present on a surface, wherein the microcapsule, through contact with an agent for washing, cleaning, conditioning, caring and/or dyeing hard or soft surfaces, comprising at least one surfactant, and a mixture of at least a first and a second microcapsule, which each comprise at least one active substance and whose capsule walls comprise a resin that is obtained by reacting a) at least one aromatic alcohol or its ethers or derivatives with b) at least one aldehydic component that possesses at least two carbon atoms per molecule, and c) optionally in the presence of at least one (meth)acrylate polymer, wherein the first and the second microcapsules differ from one another in one of the reacted components a) and/or b), is deposited onto the surface and then releases the active substance by the application of mechanical force.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was surprisingly found that the combination of selected, different formaldehyde-free microcapsules leads to synergistic effects in regard to the boost effect, storage stability and coloration in the end product.

Thus, the object was achieved by agents for washing, cleaning, conditioning, caring and/or dyeing hard or soft surfaces, comprising
i. at least one surface-active substance, and
ii. a mixture of at least a first and a second microcapsule, each of whose capsule walls comprise a resin that is obtained by reacting
   a) at least one aromatic alcohol or its ethers or derivatives with
   b) at least one aldehydic component that possesses at least two carbon atoms per molecule, and
   c) optionally in the presence of at least one (meth)acrylate polymer, wherein the first and the second microcapsules differ from one another in at least one of the reacted components a) and/or b).

Moreover, the invention relates to a method for releasing an active substance from a microcapsule that is present on a surface, wherein the microcapsule, through contact with an agent for washing, cleaning, conditioning, caring and/or dyeing hard or soft surfaces, comprising

i. at least one surface-active substance, and
ii. a mixture of at least a first and a second microcapsule, which each comprise at least one active substance and whose capsule walls comprise a resin that is obtained by reacting
d) at least one aromatic alcohol or its ethers or derivatives with
e) at least one aldehydic component that possesses at least two carbon atoms per molecule, and
f) optionally in the presence of at least one (meth)acrylate polymer, wherein the first and the second microcapsules differ from one another in at least one of the reacted components a) and/or b),
is deposited onto the surface and then releases the active substance by the application of mechanical force. The active substance is preferably released by friction.

The agents for washing, cleaning, conditioning, caring and/or dyeing hard or soft surfaces preferably comprise microcapsules in amounts of 0.0001 to 50 wt %, preferably 0.01 to 20 wt %, and especially 0.1 to 5 wt %, based on the total agent.

The first and the second microcapsules are preferably employed in the ratio 1:99 to 99:1, particularly preferably in the ratio 1:50 to 50:1 and especially preferably in the ratio 9:1 to 1:9.

In the context of this application, the agents for washing, cleaning, conditioning, caring and/or dyeing hard or soft surfaces concern washing agents, cleaning agents, post-treatment agents and/or cosmetic agents.

The agents according to the invention are used for washing, cleaning, conditioning, caring and/or dyeing hard or soft surfaces. In the context of this application, “hard surfaces” are windows, mirrors and other glass surfaces, surfaces of ceramics, plastic, metal or even wood as well as painted wood, which are found in the home and in industry, for example bathroom ceramics, kitchen and culinary dishware, kitchen surfaces or floors. In the context of this application, “soft surfaces” are textile fabrics, skin as well as hair.

In the context of this application, “agents for washing hard or soft surfaces” are fabric washing agents, e.g. in the form of powders, granules, pearls, tablets, pastes, gels, cloths, bars or liquids of the present formulations.

In the context of this application, “agents for cleaning hard or soft surfaces” include all cleaners for hard or soft surfaces, especially dishwasher detergents, all-purpose cleaners, WC cleaners, sanitary cleaners as well as glass cleaners, dental creams, skin cleansers, such as shower gels, or hair shampoos.

In the context of this application, “agents for conditioning hard or soft surfaces” are fabric softeners, fragrant rinses, conditioning cloths for use in the washer dryer, hygienic rinses, deodorants, antiperspirants, hair conditioners, styling agents and/or hair setting agents.

In the context of this application, “agents for caring for hard or soft surfaces” are fabric care agents, hair care agents or hair treatment agents, such as for example creams, lotions or gels.

In the context of this application, “agents for dyeing hard or soft surfaces” are hair dyes and hair colorants and agents for lightening keratinic fibers.

The agents for washing, cleaning, conditioning, care and/or dyeing hard or soft surfaces have the advantage that they possess at most a quite low formaldehyde content due to the fact that their manufacture involves at most a quite low, but especially no formaldehyde addition at all. The agents for washing, cleaning, conditioning, care and/or dyeing hard or soft surfaces enable the controlled release of active substances, especially fragrances and/or plant extracts, which are stored in the capsules. The capsules are stable within the agent matrix and can be opened by means of controlled stimulation, especially by mechanical force. In the context of the present invention, “mechanical force” is understood to mean any type of mechanical force on the microcapsule, such as e.g. shear forces, pressure and/or friction. In the application of the agent, e.g. in fabric washing or skin cleansing, the microcapsules are deposited on the hard or soft surfaces and after the surface has dried can be easily opened by e.g. friction. A controlled release of the active substance(s) is realized in this way, such that the performance profile of the agent as a whole is increased. In this regard, particular significance is attributed especially to the fragrance effect, as in many cases the consumer judges the product performance as a function of the pleasant odor. However, the release of the active substances, especially fragrances and/or plant extracts, can also occur by a diffusion process, in which the active substances, especially fragrances and/or care substances, migrate through the polymeric shell material and are then slowly released. The present agent for washing, cleaning, conditioning, caring and/or dyeing hard or soft surfaces enables a sustained release of active substance, in particular a sustained fragrancing and care of hard or soft surfaces as well as a controlled release, in particular the release of fragrances and/or plant extracts, even after long intervals, by employing the microencapsulated active substances.

In a preferred embodiment, the surface concerns a fabric surface. When the surface concerns a fabric surface, it is particularly preferred that the agent for washing, cleaning, conditioning, caring and/or dyeing hard or soft surfaces is a washing, cleaning or post-treatment agent.

In another embodiment, the surface concerns a body location, in particular skin and/or hair. When the surface concerns a body location, in particular skin and/or hair, it is preferred that the agent for washing, cleaning, conditioning, caring and/or dyeing hard or soft surfaces is a cosmetic composition.

The microcapsules especially comprise a liquid, preferably one or more liquid active substances. Exemplary active substances include

i. fragrances (perfume oils)
ii. care substances (e.g. plant extracts)
iii. liquid constituents of washing and cleaning agents, such as preferably surfactants, particularly non-ionic surfactants, silicone oils, paraffins
iv. liquid non-pharmaceutical additives or active substances, e.g. oils such as for example almond oil or cooling substances, as well as
v. mixtures thereof.

However, it is mostly preferred that the microcapsules comprise fragrances (perfume oils) and/or care substances. In the context of this invention, the terms “perfumes” and “fragrances” are used synonymously.

In the context of the present invention, care substances are understood to mean those substances that exert a caring effect on fabrics, the human skin and/or hair. These include plant extracts in particular. Aloe vera extract is particularly preferred.

The care substance-containing microcapsules can come into contact with the skin and/or the hair either by direct contact of the skin and/or hair with a cosmetic composition that contains care substance-containing microcapsules and/or by transmittal of the care substances and/or care substance-containing microcapsules by fabrics that carry such microcapsules on their surface.

In a preferred embodiment, the first and/or the second microcapsule comprise active substance. In this regard, the first and the second microcapsules, independently of one another, can each comprise the same or different active substances. Consequently, the active substance comprised in the first microcapsule can totally or partially differ from the active substance comprised in the second microcapsule. The first and the second microcapsules can also comprise the same active substance. Also, only the first microcapsule can comprise active substance while the second microcapsule comprises no active substance, and the other way round.

In a preferred embodiment, the added active substance concerns aloe vera extract. In a particularly preferred embodiment, at least one of the added microcapsules comprises at least 50 wt % plant extract, especially aloe vera extract, based on the total weight of the active substance comprised in the relevant microcapsules.

The usable microcapsules are described below in more detail.

In the context of the present invention, aryloxyalkanols, arylalkanols and oligoalkanol aryl ethers are preferred as the aromatic alcohols a). Aromatic compounds are likewise preferred in which at least one free hydroxy group, particularly preferably at least two free hydroxy groups, are directly bonded to the aromatic ring, wherein particularly preferably at least two free hydroxy groups are directly bonded to an aromatic ring and quite particularly preferably are meta to one another. The aromatic alcohols are preferably selected from phenols, o-cresol, m-cresol, p-cresol, α-naphthol, β-naphthol, thymol, pyrocatechol, resorcinol, hydroquinone and 1,4-naphthohydroquinone, phloroglucinol, pyrogallol, hydroxyhydroquinone and mixtures thereof.

Inventively preferred aromatic alcohols are moreover those that are used in the production of polycarbonate plastics and epoxy resin lacquers, especially 2,2-bis-(4-hydroxyphenyl)propane (Bisphenol A). The inventively present aromatic alcohol is quite particularly preferably selected from phenols with two or more hydroxy groups, preferably from pyrocatechol, resorcinol, hydroquinone and 1,4-naphthohydroquinone, phloroglucinol, pyrogallol, hydroxyhydroquinone and mixtures thereof, wherein resorcinol and/or phloroglucinol are particularly preferred as the aromatic alcohols.

In another embodiment of the present invention, the cosmetic agents comprise microcapsules, in whose manufacture the aromatic alcohol a) is added as an ether, wherein the ether in a preferred embodiment is a derivative of the relevant free form of the aromatic alcohol a) that is to be reacted. In this regard the free alcohol may also be present; therefore a mixture is present in this case. In this case the molar ratio between the free form of the aromatic alcohol to be inventively treated and the cited additional component (ether form of an aromatic alcohol) can preferably be between 0:100, preferably 1:1, or 1:2 or 1:4.

The advantage of the mixture of the aromatic alcohol with an ether form is based on the fact that the reactivity of the system can thereby be influenced. With the suitable choice of the ratio a system can be especially made, whose reactivity is appropriately attuned to the storage stability of the system. Esters are preferred as the derivatives of the aromatic alcohols.

In a preferred embodiment, the first and the second microcapsules differ from one another in the reacted components a).

Particularly stable microcapsules are obtained with the preferred aromatic alcohols a) phloroglucinol and/or resorcinol. Mixtures of microcapsules, each comprising one of these two alcohols, achieved particularly good effects in regard to the boost effect, the sedimentation behavior and the coloration in the finished product, even after protracted storage. In a particularly preferred embodiment, the first microcapsule therefore comprises phloroglucinol and the second microcapsule resorcinol as the aromatic alcohol a).

Particularly excellent results were achieved when the first microcapsule, comprising phloroglucinol, and the second microcapsule, comprising resorcinol, were employed in a ratio of 1:30 to 1:3, preferably 1:19 to 1:4 and particularly preferably 1:9 to 1:4.

According to the present invention, both aliphatic as well as aromatic aldehydes are preferred as the aldehyde b) that contains at least two carbon atoms. Particularly preferred aldehydes are one or more selected from the following group valeraldehyde, capronaldehyde, caprylaldehyde, decanal, succindialdehyde, cyclohexane carbaldehyde, cyclopentane carbaldehyde, 2-methyl-1-propanal, 2-methylpropionaldehyde, acetaldehyde, acrolein, aldosterone, antimycin A, 8′-apo-β-caroten-8′-al, benzaldehyde, butanal, chloral, citral, citronellal, crotonaldehyde, dimethylaminobenzaldehyde, folic acid, fosmidomycin, furfural, glutaraldehyde, glycerin aldehyde, glycolaldehyde, glyoxal, glyoxylic acid, heptanal, 2-hydroxybenzaldehyde, 3-hydroxybutanal, hydroxymethylfurfural, 4-hydroxynonenal, isobutanal, isobutyraldehyde, methacrolein, 2-methylundecanal, mucochloric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicyl aldehyde, secologanin, streptomycin, strophanthidin, tylosin, vanillin, cinnamaldehyde and mixtures thereof. Particularly stable microcapsules were obtained with the preferred aldehydic components b) glutardialdehyde and/or succindialdehyde.

In the context of the present invention, the aldehydic component can possess at least one or two, particularly preferably two, three or four, in particular two free aldehyde groups per molecule, wherein it is preferred when at least glyoxal, glutardialdehyde and/or succindialdehyde, particularly preferably glutardialdehyde, is present as the aldehydic component.

In the inventively usable microcapsules the molar ratio of a) the at least one aromatic alcohol or (ether or derivative thereof), to b) the at least one aldehydic component can generally be between 1:1 and 1:5, particularly preferably between 1 to 2 and 1 to 3 and quite particularly preferably for resorcinol at about 1 to 2.6. The weight ratio of the components a)+b) to c), i.e. the ratio of the sum of the weights of a)+b) to the weight of the component c) is generally between 1:1 and 1:0.01, particularly preferably between 1:0.2 and 1:0.05.

In a preferred embodiment, the first and the second microcapsules differ from one another in the reacted components b).

The optionally used (meth)acrylate polymers can be homopolymers or copolymers of methacrylate monomers and/or acrylate monomers. The term “(meth)acrylate” in this invention designates both methacrylates as well as acrylates. The (meth)acrylate polymers are e.g. homopolymers or copolymers, preferably copolymers, of one or more polar functionalized (meth)acrylate monomers, such as sulfonic acid group-containing, carboxylic acid group-containing, phosphoric acid group-containing, nitrile group-containing, phosphonic acid group-containing, ammonium group-containing, amine group-containing or nitrate group-containing (meth)acrylate monomers. In this regard, the polar groups can also be in the salt form. The (meth)acrylate polymers are suitable as protective colloids and can be advantageously used in the manufacture of microcapsules.

(Meth)acrylate copolymers can consist for example of two or more (meth)acrylate monomers (e.g. acrylate+2-acrylamido-2-methyl-propane sulfonic acid) or one or more (meth)acrylate monomers and one or more monomers that differ from (meth)acrylate monomers (e.g. methacrylate+styrene).

Exemplary (meth)acrylate polymers are homopolymers of sulfonic acid group-containing (meth)acrylates (e.g. 2-acrylamido-2-methyl-propane sulfonic acid or its salts (AMPS), commercially available as Lupasol®PA 140, BASF), or its copolymers, copolymers of acrylamide and (meth)acrylic acid, copolymers of alkyl (meth)acrylates and N-vinyl pyrrolidone (commercially available as Luviskol®K15, K30 or K90, BASF), copolymers von (meth)acrylates with polycarboxylates or polystyrene sulfonates, copolymers of (meth)acrylates with vinyl ethers and/or maleic anhydride, copolymers of (meth)acrylates with ethylene and/or maleic anhydride, copolymers of (meth)acrylates with isobutylene and/or maleic anhydride, or copolymers of (meth)acrylates with styrene-maleic anhydride.

Preferred (meth)acrylate polymers are homopolymers or copolymers, preferably copolymers, of 2-acrylamido-2-methyl-propane sulfonic acid or its salts (AMPS). Copolymers of 2-acrylamido-2-methyl-propane sulfonic acid or its salts are preferred, e.g. copolymers with one or more comonomers from the group of the (meth)acrylates, the vinyl compounds such as vinyl esters or styrenes, the unsaturated di or polycarboxylic acids such as maleic acid esters, or the salts of amyl compounds or allyl compounds. Preferred comonomers for AMPS are cited below; these comonomers can, however, also be copolymerized with other polar functionalized (meth)acrylate monomers:

1) Vinyl compounds, e.g. vinyl esters such as vinyl acetate, vinyl laurate, vinyl propionate or vinyl esters of neononanoic acid, or aromatic vinyl compounds such as styrene comonomers, for example styrene, alpha-methylstyrene or polar functionalized styrenes such as styrenes with hydroxy, amino, nitrile, carbonic acid, phosphonic acid, phosphoric acid, nitro or sulfonic acid groups and their salts, wherein the styrenes are preferably polar functionalized in the para position.
2) Unsaturated di or polycarboxylic acids, e.g. maleic acid esters such as dibutyl maleate or dioctyl maleate, as salts of allyl compounds e.g. sodium allyl sulfonate, as the salts of amyl derivatives e.g. sodium amyl sulfonate.
3) (Meth)acrylate comonomers; these are esters of acrylic acid and methacrylic acid, wherein the ester groups are e.g. saturated or unsaturated, straight chain, branched or cyclic hydrocarbon groups that can comprise one or more heteroatoms such as N, O, S, P, F, Cl, Br, I. Examples of such hydrocarbon groups are straight chain, branched or cyclic alkyl, straight chain, branched or cyclic alkenyl, aryl such as phenyl or heterocyclyl such as tetrahydrofurfuryl.

Exemplary (meth)acrylate comonomers, preferably for AMPS, are:

a) Acrylic acid, C₁-C₁₄ alkyl acrylic acids such as methacrylic acid,
b) (Meth)acrylamides such as acrylamide, methacrylamide, diacetone acrylamide, diacetone methacrylamide, N-butoxymethylacrylamide, N-iso-butoxymethylacrylamide, N-butoxymethylmethacrylamide, N-iso-butoxymethylmethacrylamide, N-methylolacrylamide, N-methylolmethacrylamide;
c) Heterocyclic (meth)acrylates such as tetrahydrofurfuryl acrylate and tetrahydrofurfuryl methacrylate or carbocyclic (meth)acrylates such as isobornyl acrylate and isobornyl methacrylate,
d) Urethane (meth)acrylates such as diurethane diacrylate and diurethane methacrylate (CAS: 72869-86-4)
e) C₁-C₁₄ alkyl acrylates such as methyl, ethyl, n-propyl, isopropyl, n-butyl, sec. butyl, iso-butyl, tert. butyl, n-pentyl, iso-pentyl, hexyl (e.g. n-hexyl, iso-hexyl or cyclohexyl), heptyl, octyl (e.g. 2-ethylhexyl), nonyl, decyl (e.g. 2-propylheptyl or iso-decyl), undecyl, dodecyl, tridecyl (e.g. iso-tridecyl), and tetradecyl acrylate; the alkyl groups can optionally be substituted with one or more halogen atoms (e.g. fluorine, chlorine, bromine or iodine), e.g. trifluoroethyl acrylate, or with one or more amino groups, e.g. diethylaminoethyl acrylate, or with one or more alkoxy groups such as methoxypropyl acrylate, or with one or more aryloxy groups such as phenoxyethyl acrylate.
f) C₂-C₁₄ alkenyl acrylates such as ethenyl, n-propenyl, isopropenyl, n-butenyl, sec. butenyl, iso-butenyl, tert. butenyl, n-pentenyl, iso-pentenyl, hexenyl (e.g. n-hexenyl, iso-hexenyl or cyclohexenyl), heptenyl, octenyl (e.g. 2-ethylhexenyl), nonenyl, decenyl (e.g. 2-propenylheptyl or iso-decenyl), undecenyl, dodecenyl, tridecenyl (e.g. iso-tridecenyl), and tetradecenyl acrylate, and their epoxides such as glycidyl acrylate or aziridines such as aziridine acrylate.
g) C₁-C₁₄ hydroxyalkyl acrylates such as hydroxymethyl, hydroxyethyl, hydroxy-n-propyl, hydroxy-isopropyl, hydroxy-n-butyl, hydroxy-sec.-butyl, hydroxy-iso-butyl, hydroxy-tert.-butyl, hydroxy-n-pentyl, hydroxy-iso-pentyl, hydroxyhexyl (e.g. hydroxy-n-hexyl, hydroxy-iso-hexyl or hydroxy-cyclohexyl), hydroxyheptyl, hydroxyoctyl (e.g. 2-ethylhexyl), hydroxynonyl, hydroxydecyl (e.g. hydroxy-2-propylheptyl or hydroxy-iso-decyl), hydroxyundecyl, hydroxydodecyl, hydroxytridecyl (e.g. hydroxy-iso-tridecyl), and hydroxytetradecyl acrylate, wherein the hydroxy group of the alkyl group is preferably in the terminal position (ω-position) (e.g. 4-hydroxy-n-butyl acrylate) or in the (ω-1)-position (e.g. 2-hydroxy-n-propyl acrylate);
h) Alkylene glycol acrylates, which comprise one or more alkylene glycol units. Examples are i) monoalkylene glycol acrylates, such as the acrylates of ethylene glycol, propylene glycol (e.g. 1,2- or 1,3-propane diol), butylene glycol (e.g. 1,2-, 1,3- or 1,4-butane diol, pentylene glycol (e.g. 1,5-pentane diol) or hexylene glycol (e.g. 1,6-hexane diol), in which the second hydroxy group is etherified or esterified, e.g. by sulfuric acid, phosphoric acid, acrylic acid or methacrylic acid, or ii) polyalkylene glycol acrylates such as polyethylene glycol acrylates, polypropylene glycol acrylates, polybutylene glycol acrylates, polypentylene glycol acrylates or polyhexylene glycol acrylates, whose second hydroxy group can be optionally etherified or esterified, e.g. by sulfuric acid, phosphoric acid, acrylic acid or methacrylic acid.

Examples of (poly)alkylene glycol units with etherified hydroxy groups are C₁-C₁₄ alkyloxy-(poly)alkylene glycols (e.g. C₁-C₁₄ alkyloxy-polyalkylene glycol acrylates), examples of (poly)alkylene glycol units with esterified hydroxy groups are sulfonium-(poly)alkylene glycols (e.g. sulfonium-(poly)alkylene glycol acrylates) and their salts, (poly)alkylene glycol diacrylates such as 1,4-butane diol diacrylate or 1,6-hexane diol diacrylate or (poly)alkylene glycol methacrylate acrylates such as 1,4-butane diol methacrylate acrylate or 1,6-hexane diol methacrylate acrylate.

The polyalkylene glycol acrylates can carry an acrylate group (e.g. polyethylene glycol monoacrylate, polypropylene glycol monoacrylate, polybutylene glycol monoacrylate, polypentylene glycol monoacrylate or polyhexylene glycol monoacrylate) or two or more, preferably two, acrylate groups such as polyethylene glycol diacrylate, polypropylene glycol diacrylate, polybutylene glycol diacrylate, polypentylene glycol diacrylate or polyhexylene glycol diacrylate.

The polyalkylene glycol acrylates can also comprise two or more polyalkylene glycol blocks that differ from each other, e.g. blocks of polymethylene glycol and polyethylene glycol or blocks of polyethylene glycol and polypropylene glycol.

The degree of polymerization of the polyalkylene glycol units or polyalkylene glycol blocks is generally in the range of 1 to 20, preferably in the range 3 to 10, particularly preferably in the range of 3 to 6.

i) C₁-C₁₄ alkyl methacrylates such as methyl, ethyl, n-propyl, isopropyl, n-butyl, sec. butyl, iso-butyl, tert. butyl, n-pentyl, iso-pentyl, hexyl (e.g. n-hexyl, iso-hexyl or cyclohexyl), heptyl, octyl (e.g. 2-ethylhexyl), nonyl, decyl (e.g. 2-propylheptyl or iso-decyl), undecyl, dodecyl, tridecyl (e.g. iso-tridecyl), and tetradecyl methacrylate; the alkyl groups can optionally be substituted with one or more halogen atoms (e.g. fluorine, chlorine, bromine or iodine), e.g. trifluoroethyl methacrylate, or with one or more amino groups, e.g. diethylaminoethyl methacrylate, or with one or more alkoxy groups such as methoxypropyl methacrylate, or with one or more aryloxy groups such as phenoxyethyl methacrylate;
j) C₂-C₁₄ alkenyl methacrylates such as ethenyl, n-propenyl, isopropenyl, n-butenyl, sec. butenyl, iso-butenyl, tert. butenyl, n-pentenyl, iso-pentenyl, hexenyl (e.g. n-hexenyl, iso-hexenyl or cyclohexenyl), heptenyl, octenyl (e.g. 2-ethylhexenyl), nonenyl, decenyl (e.g. 2-propenylheptyl or iso-decenyl), undecenyl, dodecenyl, tridecenyl (e.g. iso-tridecenyl), and tetradecenyl methacrylate, and their epoxides such as glycidyl methacrylate or aziridines such as aziridine methacrylate;
k) C₁-C₁₄ hydroxyalkyl methacrylates such as hydroxymethyl, hydroxyethyl, hydroxy-n-propyl, hydroxy-isopropyl, hydroxy-n-butyl, hydroxy-sec.-butyl, hydroxy-iso-butyl, hydroxy-tert.-butyl, hydroxy-n-pentyl, hydroxy-iso-pentyl, hydroxyhexyl (e.g. hydroxy-n-hexyl, hydroxy-iso-hexyl or hydroxy-cyclohexyl), hydroxyheptyl, hydroxyoctyl (e.g. 2-ethylhexyl), hydroxynonyl, hydroxydecyl (e.g. hydroxy-2-propylheptyl or hydroxy-iso-decyl), hydroxyundecyl, hydroxydodecyl, hydroxytridecyl (e.g. hydroxy-iso-tridecyl), and hydroxytetradecyl methacrylate, wherein the hydroxy group of the alkyl group is preferably in the terminal position (w-position) (e.g. 4-hydroxy-n-butyl methacrylate) or in the (ω-1)-position (e.g. 2-hydroxy-n-propyl methacrylate);
1) Alkylene glycol methacrylates that comprise one or more alkylene glycol units. Examples are i) monoalkylene glycol methacrylates, such as methacrylates of ethylene glycol, propylene glycol (e.g. 1,2- or 1,3-propane diol), butylene glycol (e.g. 1,2-, 1,3- or 1,4-butane diol, pentylene glycol (e.g. 1,5-pentane diol) or hexylene glycol (e.g. 1,6-hexane diol), in which the second hydroxy group is etherified or esterified, e.g. by sulfuric acid, phosphoric acid, acrylic acid or methacrylic acid, or ii) polyalkylene glycol methacrylates such as polyethylene glycol methacrylates, polypropylene glycol methacrylates, polybutylene glycol methacrylates, polypentylene glycol methacrylates or polyhexylene glycol methacrylates, whose second hydroxy group can be optionally etherified or esterified, e.g. by sulfuric acid, phosphoric acid, acrylic acid or methacrylic acid.

Examples of (poly)alkylene glycol units with etherified hydroxyl groups are C₁-C₁₄ alkyloxy (poly)alkylene glycols (e.g. C₁-C₁₄ alkyloxy polyalkylene glycol methacrylates), examples of (poly)alkylene glycol units with esterified hydroxy groups are sulfonium-(poly)alkylene glycols (e.g. sulfonium-(poly)alkylene glycol methacrylates) and their salts or (poly)alkylene glycol dimethacrylates such as 1,4-butane diol dimethacrylate.

The polyalkylene glycol methacrylates can carry a methacrylate group (e.g. polyethylene glycol monomethacrylate, polypropylene glycol monomethacrylate, polybutylene glycol monomethacrylate, polypentylene glycol monomethacrylate or polyhexylene glycol monomethacrylate) or two or more, preferably two, methacrylate groups such as polyethylene glycol dimethacrylate, polypropylene glycol dimethacrylate, polybutylene glycol dimethacrylate, polypentylene glycol dimethacrylate or polyhexylene glycol dimethacrylate.

The polyalkylene glycol methacrylates can also comprise two or more polyalkylene glycol blocks that differ from each other, e.g. blocks of polymethylene glycol and polyethylene glycol or blocks of polyethylene glycol and polypropylene glycol (e.g. Bisomer PEM63PHD (Cognis), CAS 58916-75-9).

The degree of polymerization of the polyalkylene glycol units or polyalkylene glycol blocks is generally in the range of 1 to 20, preferably in the range 3 to 10, particularly preferably in the range of 3 to 6.

Exemplary preferred (meth)acrylate comonomers are 4-hydroxybutyl acrylate, 2-hydroxypropyl methacrylate, ammonium sulfatoethyl methacrylate, pentapropylene glycol methacrylate, acrylic acid, hexaethylene glycol methyacrylate, hexapropylene glycol acrylate, hexaethylene glycol acrylate, hydroxyethyl methacrylate, polyalkylene glycol methacrylate (CAS-Nr. 589-75-9), Bisomer PEM63PHD, methoxy polyethylene glycol methacrylate, 2-propylheptyl acrylate (2-PHA), 1,3-butane diol dimethacrylate (BDDMA), triethylene glycol dimethacrylate (TEGDMA), hydroxyethyl acrylate (HEA), 2-hydroxypropyl acrylate (HPA), ethylene glycol dimethacrylate (EGDMA), glycidyl methacrylate (GMA) and/or allyl methacrylate (ALMA).

The AMPS copolymers generally have a fraction of AMPS units that is greater than 50 mol %, preferably in the range 60 to 95 mol %, particularly preferably 80 to 99 mol %, the fraction of comonomers generally being less than 50 mol %, preferably in the range 5 to 40 mol %, particularly preferably 1 to 20 mol %.

The copolymers can be obtained by processes known per se, for example in batch or semi-batch processes. For example, appropriate amounts of water and monomers are first fed into a temperature controlled reactor and maintained under an atmosphere of inert gas. The mixture is then brought to the reaction temperature with stirring (preferably 70 to 80° C.), initiator is added, preferably in the form of an aqueous solution. Suitable initiators are the known initiators for radical polymerizations, for example the peroxydisulfates of sodium, potassium or ammonium, or mixtures based on H₂O₂, e.g. mixtures of H₂O₂ with citric acid. Once the maximum temperature has been attained and as the temperature in the reactor starts to fall either a) the remaining monomers are added and subsequently reacted (semi-batch process), or b) the next reaction is carried out directly (batch process). The resulting reaction mixture is then cooled down to room temperature and the copolymer is isolated from the aqueous solution, e.g. by extraction with organic solvents such as hexane or methylene chloride and subsequent distillation of the solvent. The copolymer can then be washed with organic solvent and dried. The reaction mixture can also be directly processed; in this case it is advantageous to add a preservative to the aqueous copolymer solution.

The AMPS copolymers are suitable as protective colloids for manufacturing the microcapsules that are usable according to the present invention.

The microcapsules or microcapsule dispersions that are comprised in the inventive washing, cleaning, conditioning, caring and/or dyeing agents for hard or soft surfaces are preferably manufactured by reacting together the at least one inventively reactive aromatic alcohol and the at least one inventively reactive aldehyde component that possesses at least two carbon atoms per molecule, optionally in the presence of at least one (meth)acrylate polymer, optionally in the presence of a substance to be encapsulated (core material) whereupon the capsules are subsequently cured by increasing the temperature. It is particularly preferred in this regard to increase the pH in the course of the process.

Preferably, in the context of such a process, initially

a) the at least one aromatic alcohol and/or its derivative or ether and the at least one aldehydic component and optionally at least one (meth)acrylate polymer and at least one substance to be encapsulated are brought together at a temperature of 40 to 65° C. and a pH between 6 and 9, preferably 7 and 8.5, and
b) in a later process step at a temperature of 40 to 65° C. the pH is increased to more than 9, preferably between 9.5 and 11, wherein
c) the capsules are then cured by increasing the temperature to 60 to 110° C., preferably 70 to 90° C., especially 80° C.

However, if phloroglucinol is used as the alcohol component, then curing is more advantageously carried out under acidic conditions; preferably the pH is then maximum 4, particularly preferably between 3 and 4, for example between 3.2 and 3.5.

The resulting capsules are free of formaldehyde and as the stable core/shell microcapsules can be processed without problems from the aqueous slurry to a dry free-flowing powder.

In general, the capsules can be loaded with gaseous, liquid as well as solid materials. Hydrophobic materials are preferably incorporated. However, liquid substances, active substances, especially fragrances and plant extracts are particularly preferred, as well as surfactants, especially non-ionic surfactants, silicone oils, paraffins, liquid non-pharmaceutical additives or active substances, e.g. oils such as for example almond oil as well as mixtures of the above. However, it is most preferred that the microcapsules comprise fragrances (perfume oils) and/or plant extracts.

As fragrances or perfumes or perfume oils, all substances and mixtures known as these can be used. In the context of this invention, the terms “perfume(s)”, “fragrances” and “perfume oil(s)” are used synonymously. In particular, they mean any substances or their mixtures that are perceived by humans and animals as an odor, in particular by humans as a pleasant odor.

Perfumes, perfume oils or constituents of perfume oils can be employed as the fragrant components. According to the invention perfume oils or fragrances can be individual fragrant compounds, for example the synthetic products of the ester, ether, aldehyde, ketone, alcohol and hydrocarbon type. Fragrant compounds of the ester type are, for example, benzyl acetate, phenoxyethyl isobutyrate, p-tert.-butylcyclohexyl acetate, linalyl acetate, dimethylbenzyl carbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate and jasmecyclate. The ethers include, for example, benzyl ethyl ether and ambroxan; the aldehydes include, for example, the linear alkanals containing 8 to 18 carbon atoms, citral, citronellal, citronellyloxyacetaldehyde, cyclamen aldehyde, lilial and bourgeonal; the ketones include, for example, the ionones, alpha-isomethyl ionone and methyl cedryl ketone; the alcohols include anethol, citronellol, eugenol, geraniol, linalool, phenylethyl alcohol and terpineol and the hydrocarbons include, in particular the terpenes, such as limonene and pinene. However, mixtures of various fragrant substances, which together produce an attractive fragrant note, are preferably used.

Perfume oils such as these may also contain natural mixtures of fragrant substances, as are obtainable from vegetal sources, for example pine, citrus, jasmine, patchouli, rose or ylang-ylang oil. Also suitable are muscatel sage oil, chamomile oil, clove oil, melissa oil, mint oil, cinnamon leaf oil, lime blossom oil, juniper berry oil, vetivert oil, olibanum oil, galbanum oil and laudanum oil as well as orange blossom oil, neroli oil, orange peel oil and sandalwood oil.

The volatility of a fragrant substance is crucial for its perceptibility, whereby in addition to the nature of the functional groups and the structure of the chemical compound, the molecular weight also plays an important role. Thus, the majority of fragrant substances have molecular weights up to about 200 daltons, whereas molecular weights of 300 daltons and above are quite an exception. Due to the different volatilities of fragrant substances, the smell of a perfume or fragrance composed of a plurality of fragrant substances changes during evaporation, the impressions of odor being subdivided into the “top note”, “middle note” or “body” and “end note” or “dry out”. As the perception of smell also depends to a large extent on the intensity of the odor, the top note of a perfume or fragrance consists not solely of highly volatile compounds, whereas the end note consists to a large extent of less volatile, i.e. tenacious fragrant substances. In the composition of perfumes, higher volatile fragrant substances can be bound, for example onto particular fixatives, whereby their rapid evaporation is impeded. In the following subdivision of fragrant substances into “more volatile” or “tenacious” fragrant substances, nothing is mentioned about the odor impression and further, whether the relevant fragrant substance is perceived as the top note or body note. Exemplary tenacious odorous substances that can be used in the context of the present invention are the ethereal oils such as angelica root oil, aniseed oil, arnica flowers oil, basil oil, bay oil, bergamot oil, champax blossom oil, silver fir oil, silver fir cone oil, elemi oil, eucalyptus oil, fennel oil, pine needle oil, galbanum oil, geranium oil, ginger grass oil, guaiacum wood oil, Indian wood oil, helichrysum oil, ho oil, ginger oil, iris oil, cajuput oil, sweet flag oil, camomile oil, camphor oil, Canoga oil, cardamom oil, cassia oil, Scotch fir oil, copaiba balsam oil, coriander oil, spearmint oil, caraway oil, cumin oil, lavender oil, lemon grass oil, limette oil, mandarin oil, melissa oil, amber seed oil, myrrh oil, clove oil, neroli oil, niaouli oil, olibanum oil, orange oil, origanum oil, Palma Rosa oil, patchouli oil, Peru balsam oil, petit grain oil, pepper oil, peppermint oil, pimento oil, pine oil, rose oil, rosemary oil, sandalwood oil, celery seed oil, lavender spike oil, Japanese anise oil, turpentine oil, thuja oil, thyme oil, verbena oil, vetiver oil, juniper berry oil, wormwood oil, wintergreen oil, ylang-ylang oil, ysop oil, cinnamon oil, cinnamon leaf oil, citronella oil, citrus oil and cypress oil. However, in the context of the present invention, the higher boiling or solid fragrant substances of natural or synthetic origin can be used as tenacious fragrant substances or mixtures thereof, namely fragrances. These compounds include the following compounds and their mixtures: ambrettolide, α-amyl cinnamaldehyde, anethol, anisaldehyde, anis alcohol, anisole, methyl anthranilate, acetophenone, benzyl acetone, benzaldehyde, ethyl benzoate, benzophenone, benzyl alcohol, benzyl acetate, benzyl benzoate, benzyl formate, benzyl valeriate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, methyl heptynecarboxylate, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvacrol, carvone, p-cresol methyl ether, coumarone, p-methoxyacetophenone, methyl-n-amyl ketone, methylanthranilic acid methyl ester, p-methyl acetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenylethyl alcohol, phenyl acetaldehyde dimethyl acetal, phenyl acetic acid, pulegone, safrol, isoamyl salicylate, methyl salicylate, hexyl salicylate, cyclohexyl salicylate, santalol, scatol, terpineol, thymine, thymol, γ-undecalactone, vanillin, veratrum aldehyde, cinnamaldehyde, cinnamyl alcohol, cinnamic acid, ethyl cinnamate, benzyl cinnamate.

The readily volatile fragrant substances particularly include the low boiling fragrant substances of natural or synthetic origin that can be used alone or in mixtures. Exemplary readily volatile fragrant substances are alkyl isothiocyanates (alkyl mustard oils), butanedione, limonene, linalool, linalyl acetate and linalyl propionate, menthol, menthone, methyl n-heptenone, phellandrene, phenyl acetaldehyde, terpinyl acetate, citral, citronellal.

Preferred usable (especially for encapsulation) fragrant compounds of the aldehyde type are hydroxycitronellal (CAS 107-75-5), helional (CAS 1205-17-0), citral (5392-40-5), bourgeonal (18127-01-0), triplal (CAS 27939-60-2), ligustral (CAS 68039-48-5), vertocitral (CAS 68039-49-6), florhydral (CAS 125109-85-5), citronellal (CAS 106-23-0), citronellyloxyacetaldehyde (CAS 7492-67-3).

It is further preferred that the perfume to be encapsulated does not include 2-methyl-undecanal, decanal, benzeneacetaldehyde or 3-phenylprop-2-enal.

The microcapsules can preferably also comprise one or more (preferably liquid) skin care and/or skin protecting active substances. Skin-care active principles are all such active principles that lend a sensorial and/or cosmetic advantage to the skin. Skin-care active principles are preferably selected from the following substances:

a) waxes such as for example carnauba, spermaceti, beeswax, lanoline and/or derivatives of the same and others.
b) hydrophobic plant extracts
c) hydrocarbons such as for example squalene and/or squalane
d) higher fatty acids, preferably those containing at least 12 carbon atoms, for example lauric acid, stearic acid, behenic acid, myristic acid, palmitic acid, oleic acid, linoleic acid, linolenic acid, isostearic acid and/or polyunsaturated fatty acids and others.
e) higher fatty alcohols, preferably those containing at least 12 carbon atoms, for example lauryl alcohol, cetyl alcohol, stearyl alcohol, oleyl alcohol, behenyl alcohol, cholesterol and/or 2-hexadecanol and others.
f) esters, preferably those such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactates, alkyl citrates and/or alkyl tartrates and others.
g) lipids such as for example cholesterol, ceramides and/or saccharose esters and others.
h) vitamins such as for example vitamins A, C and E, vitamin alkyl esters, including vitamin C alkyl esters and others.
i) sunscreens
j) phospholipids
k) derivatives of alpha-hydroxyacids
l) germicides for cosmetic use, both synthetic, such as salicylic acid and/or others, as well as naturally occurring, such as for example neem oil and/or others.
m) silicones
n) naturally occurring oils, e.g. almond oil
as well as mixtures of any of the above components.

The microcapsules preferably comprise plant extracts as the active substance. Usually, these extracts are manufactured by extraction of the whole plant. In individual cases, however, it can also be preferred to produce the extracts solely from blossoms and/or leaves of the plant.

According to the invention, mainly extracts from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred. Extracts of aloe vera are especially preferred.

The extraction composition used to prepare the cited plant extracts can be water, alcohols as well as their mixtures. Exemplary preferred alcohols are lower alcohols such as ethanol and isopropanol, but particularly polyhydric alcohols such as ethylene glycol and propylene glycol, both as the sole extraction agent as well as in aqueous mixtures. Plant extracts based on e.g. water/propylene glycol in proportions of 1:10 to 10:1 have proven to be particularly suitable.

According to the invention, the plant extracts can be used in pure as well as in diluted form. When they are used in diluted form, they normally comprise ca. 2 to 80% by weight active substance and the solvent is the extraction agent or mixture of extraction agents used for their extraction.

Furthermore, it can be preferred to employ a plurality, especially two different plant extracts as the active substance. Moreover, perfume oils and plant extracts can be employed together as the active substance.

In general, the diameter of the microcapsules is in the range 1 to 1000 μm. In the context of the present invention, the term “microcapsule” also includes nanocapsules, i.e. capsules with a diameter <1 μm. The average diameter of the capsules is preferably from 0.1 to 100 The wall thickness can be 0.05 to 10 μm for example.

The claimed agents for washing, cleaning, conditioning, caring and/or dyeing hard or soft surfaces comprise, in addition to the described microcapsules, still other ingredients, namely at least surface active substances.

Anionic surfactants, non-ionic surfactants, cationic, zwitterionic and/or amphoteric surfactants and/or emulsifiers are especially considered as the surface active substances. However, it is particularly preferred when the agent for washing, cleaning, conditioning, caring and/or dyeing hard or soft surfaces comprises anionic, non-ionic and/or cationic surfactants. The use of a mixture of anionic and non-ionic surfactants is particularly advantageous. The agent preferably comprises 0.05 to 50 wt %, more advantageously 1 to 40 wt %, still more advantageously 3 to 30 wt % and in particular 5 to 20 wt % surface active substance, in particular from the group of the anionic surfactants, non-ionic surfactants, cationic, zwitterionic, amphoteric surfactants and/or emulsifiers. This corresponds to a preferred embodiment of the invention and provides optimal cleaning powers.

It is particularly preferred when the agent for washing, cleaning, conditioning, caring and/or dyeing hard or soft surfaces comprises anionic surfactant, advantageously in amounts of 0.1 to 25 wt %, more advantageously 1 to 20 wt %, in particular in amounts of 3 to 15 wt %, relative to the total agent. This corresponds to a preferred embodiment of the invention and provides particularly advantageous cleaning powers. A particularly suitable anionic surfactant is alkylbenzene sulfonate, preferably linear alkylbenzene sulfonate (LAS). If the agent comprises alkylbenzene sulfonate, advantageously in amounts of 0.1 to 25 wt %, more advantageously 1 to 20 wt %, in particular in amounts of 3 to 15 wt %, relative to the total agent, then this is a preferred embodiment of the invention.

Further particularly suitable anionic surfactants are the alkyl sulfates, in particular the fatty alcohol sulfates (FAS), such as e.g. C₁₂-C₁₈ fatty alcohol sulfate. C₈-C₁₈ Alkyl sulfates can preferably be added, C₁₃ alkyl sulfate as well as C₁₃₋₁₅ alkyl sulfate and C₁₃₋₁₇ alkyl sulfate are particularly preferred, advantageously branched, especially alkyl-branched C₁₃₋₁₇ alkyl sulfate. Particularly suitable fatty alcohol sulfates are derived from lauryl alcohol and myristyl alcohol and are therefore fatty alcohol sulfates with 12 or 14 carbon atoms. The long chain FAS-types (C₁₆ to C₁₈) are very well suited for washing at higher temperatures. Alkyl sulfates with a low Krafft temperature, preferably with a Krafft temperature below 45, 40, 30 or 20° C., are particularly preferred.

The Krafft temperature designates that temperature at which the solubility of surfactants strongly increases as a result of the formation of micelles. The Krafft temperature is a triple point, at which the solid or hydrated crystals of the surfactant are in equilibrium with the dissolved (hydrated) monomers and micelles. The Krafft temperature is determined by a turbidity measurement according to DIN EN 13955: 2003. When the agent for washing, cleaning, conditioning, caring and/or dyeing hard or soft surfaces comprises alkyl sulfate, especially C₁₂-C₁₈ fatty alcohol sulfate, advantageously in amounts of 0.1 to 25 wt %, more advantageously 1 to 20 wt %, in particular in amounts of 3 to 15 wt %, relative to the total agent, then this is a preferred embodiment of the invention.

Other preferred anionic surfactants that can be used are e.g. alkane sulfonates (e.g. secondary C₁₃-C₁₈ alkane sulfonate), methyl ester sulfonates (e.g. α-C₁₂-C₁₈ methyl ester sulfonate) and α-olefin sulfonates (e.g. α-C₁₄-C₁₈ olefin sulfonate) and alkyl ether sulfates (e.g. C12-C14 fatty alcohol-2EO-ether sulfate) and/or soaps. Further suitable anionic surfactants will be described further below. However, FAS and/or LAS are particularly suitable.

The anionic surfactants, including the soaps, may be in the form of their sodium, potassium or ammonium salts or as soluble salts of organic bases, such as mono, di or triethanolamine. Preferably, the anionic surfactants are in the form of their sodium or potassium salts, especially in the form of the sodium salts.

It is particularly preferred when the agent for washing, cleaning, conditioning, caring and/or dyeing hard or soft surfaces comprises non-ionic surfactant, advantageously in amounts of 0.01 to 25 wt %, more advantageously 1 to 20 wt %, in particular in amounts of 3 to 15 wt %, relative to the total agent. This corresponds to a preferred embodiment of the invention. The use of alkyl polyglycol ethers is particularly preferred, in particular in combination with anionic surfactant, such as preferably LAS.

Further suitable non-ionic surfactants are alkylphenol polyglycol ethers (APEO), (ethoxylated) sorbitol fatty acid esters (sorbitanes), alkyl polyglucosides (APG), fatty acid glucamides, fatty acid ethoxylates, amine oxides, ethylene oxide-propylene oxide block polymers, polyglycerol fatty acid esters and/or fatty acid alkanolamides. Further suitable non-ionic surfactants will be described further below. Non-ionic surfactants based on sugars, such as especially APG, are particularly preferred.

In another preferred embodiment the surface active substances are emulsifiers. Emulsifiers act at the interface to produce water or oil-stable adsorption layers that protect the dispersed droplets against coalescence and thereby stabilize the emulsion. Thus, emulsifiers, like surfactants, are composed of hydrophobic and hydrophilic molecular moieties. Hydrophilic emulsifiers preferably form O/W emulsions and hydrophobic emulsifiers preferably form W/O emulsions. An emulsion is understood to mean a dispersion of a liquid in the form of droplets in another liquid using an energy input to afford interfaces stabilized with surfactants. The choice of this emulsifying surfactant or emulsifier depends on the materials being dispersed and the respective external phase as well as the fineness of the emulsion. Exemplary inventively usable emulsifiers are
Addition products of 4 to 30 moles ethylene oxide and/or 0 to 5 moles propylene oxide to linear fatty alcohols containing 8 to 22 carbon atoms, to fatty acids containing 12 to 22 carbon atoms and to alkyl phenols containing 8 to 15 carbon atoms in the alkyl group,
C12 to C22 fatty acid mono and diesters of addition products of 1 to 30 moles ethylene oxide to polyols with 3 to 6 carbon atoms, particularly glycerin,
ethylene oxide and polyglycerin addition products on methyl glucoside fatty acid esters, fatty acid alkanolamides and fatty acid glucamides,
C8 to C22 alkyl mono and oligoglycosides and their ethoxylated analogs, wherein the degrees of oligomerization are 1.1 to 5, particularly 1.2 to 2.0, and glucose as the sugar component are preferred,
Addition products of 5 to 60 mole ethylene oxide onto castor oil and hydrogenated castor oil,
Partial esters of polyols containing 3 to 6 carbon atoms with saturated fatty acids containing 8 to 22 carbon atoms,
Sterols. Sterols are understood to mean a group of steroids, which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
Phospholipids. Among these are principally meant the glucose-phospholipids, which are obtained e.g. as lecithins or phosphatidyl cholines from e.g. egg yolk or plant seeds (e.g. soya beans).
Fatty acid esters of sugars and sugar alcohols such as sorbitol,
Polyglycerins and polyglycerin derivatives such as for example polyglycerin poly-12-hydroxystearate (commercial product Dehymuls® PGPH),
Linear and branched fatty acids with 8 to 30 carbon atoms and their Na, K, ammonium, Ca, Mg and Zn salts.

When the agent for washing, cleaning, conditioning, caring and/or dyeing hard or soft surfaces is a washing, cleaning or post-treatment agent, then in addition to the essential ingredients, it can comprise additional ingredients that further improve the application and/or aesthetic properties of the washing, cleaning or post-treatment agent. In the context of the present invention, the washing, cleaning or post-treatment agent preferably additionally comprises one or a plurality of materials from the group of the builders, bleaching agents, bleach catalysts, bleach activators, enzymes, electrolytes, non-aqueous solvents, pH adjustors, perfume compositions, perfume carriers, fluorescent agents, dyes, hydrotropes, foam inhibitors, silicone oils, soil-release polymers, graying inhibitors, shrink preventers, anti-crease agents, color transfer inhibitors, additional antimicrobials, germicides, fungicides, antioxidants, preservatives, corrosion inhibitors, antistats, bittering agents, ironing aids, water-repellents and impregnation agents, swelling and non-skid agents, softening components and UV-absorbers.

Particularly preferred additional ingredients for washing, cleaning or post-treatment agents are builders, enzymes, electrolytes, non-aqueous solvents, pH adjustors, perfume compositions, fluorescent agents, dyes, hydrotropes, foam inhibitors, soil-release polymers, graying inhibitors, color transfer inhibitors, softening components, UV-absorbers as well as mixtures thereof.

In a preferred embodiment, the washing, cleaning or post-treatment agents are in liquid form and comprise water as the principal solvent.

The invention also relates to the use of a washing, cleaning or post-treatment agent for washing, cleaning and/or pre-treating textile fabrics.

When the agent for washing, cleaning, conditioning, caring and/or dyeing hard or soft surfaces is a cosmetic composition then it can comprise further ingredients in addition to the essential constituents. The cosmetic composition preferably additionally comprises at least one cosmetic active substance from the group of the oxidation dye precursors, the substantive dyes the oxidizing agents selected from hydrogen peroxide and its addition products on solid carriers, the hair conditioning active substances, the deodorant and/or antiperspirant active substances, the skin lightening and/or skin soothing and/or moisture-donating actives, the inorganic and/or organic UV filtering substances, the sebum regulators, the mechanical exfoliators, the antimicrobials, the hair setting or hair styling actives, the anti-caries active substances, the tartar inhibitors and the mixtures of these active substances. These cosmetic active substances are preferably comprised in 0.01 up to 70 wt %, based on the total weight of the ready for use agent.

Another subject matter of the invention is a method for producing a liquid agent for agent for washing, cleaning, conditioning, caring and/or dyeing hard or soft surfaces, characterized by stirring a dispersion of microcapsules, containing at least a first and a second microcapsule, whose capsule walls contain a resin that is obtained by reacting

a) at least one aromatic alcohol or its ethers or derivatives with
b) at least one aldehydic component that possesses at least two carbon atoms per molecule, and
c) optionally in the presence of at least one (meth)acrylate polymer, into the liquid matrix of the agent for washing, cleaning, conditioning, caring and/or dyeing hard or soft surfaces or by continuously adding the cited dispersion of microcapsules into a liquid matrix and blending through a static mixer element, wherein surfactant was preferably added beforehand to each of the dispersions of microcapsules, wherein the first and the second microcapsules differ from one another in at least one of the reacted components a) and/or b).

Another subject matter of the invention is a method for producing a solid agent for agent for washing, cleaning, conditioning, caring and/or dyeing hard or soft surfaces, characterized

(i) by blending a microcapsule dispersion, containing at least a first and a second microcapsule, whose capsule walls contain a resin that is obtainable by treating
a) at least one aromatic alcohol or its ethers or derivatives with
b) at least one aldehydic component that possesses at least two carbon atoms per molecule, and
c) optionally in the presence of at least one (meth)acrylate polymer, into the usual matrix of the agent for washing, cleaning, conditioning, caring and/or dyeing hard or soft surfaces, or
(ii) by blending the cited microcapsules in granulated or supported form into the usual matrix, or
(iii) by mixing the cited microcapsules in dried form into the usual matrix, wherein the first and the second microcapsule differ from one another in at least one of the reacted components a) and/or b).

### EXAMPLES

### I. Synthesis Examples

### Example I.1

### Production of Copolymers

a) AMPS-Hydroxybutyl Acrylate

For the 1500 g batch, 891 g of deionized water together with 585 g AMPS (50% aqueous solution) and 7.5 g 4-hydroxybutyl acrylate (HBA) were fed into the reactor and placed under an atmosphere of inert gas. The reaction mixture was heated to 75° C. with stirring (400 rpm). When the mixture reached the reaction temperature, 0.03 g of the water-soluble initiator sodium peroxydisulfate, dissolved in 15 g water, was injected into the reactor by means of a syringe. After the maximum temperature was attained, there began an hour of continued reaction. The batch was then cooled down to room temperature and 1.5 g of preservative was added.

The aqueous solution was characterized by its viscosity, solids content and the pH. The viscosity was 540 mPas (Brookfield measured at 20 rpm), the solids content was 21% and the pH was 3.3. 3 g of copolymer were deposited on a Petri dish and dried for 24 hours at 160° C. in the drying oven. The resulting weight was 0.69 g, corresponding to a yield of 21.6%.

b) AMPS-Polyalkylene Glycol Monomethacrylate

The reaction mixture consisted of 912 g deionized water, 240 g AMPS and 7.5 g poly(ethylene/propylene) glycol monomethacrylate (Bisomer PEM 63P HD from Cognis, CAS-Nr. 589-75-9). The mixture was placed under an atmosphere of inert gas. The reaction mixture was heated to 75° C. with stirring (400 rpm). Into the reactor was injected a solution of 1.5 g sodium peroxydisulfate in 15 g water by means of a syringe. Once the temperature in the reactor had reached a maximum and had begun to drop, 240 g AMPS with 83 g PEM 63P HD were metered in by means of a peristaltic pump over a period of one hour. The reaction was then allowed to proceed for half an hour. The batch was then cooled down to room temperature and 1.5 g of preservative was added.

The aqueous solution was characterized by its viscosity, solids content and the pH. The viscosity was 110 mPas (Brookfield measured at 20 rpm), the solids content was 23% and the pH was 3.1. 3 g of copolymer were deposited on a Petri dish and dried for 24 hours at 160° C. in the drying oven. The resulting weight was 0.68 g, corresponding to a yield of 21.6%.

### 1. Example I.2

### Resorcinol Capsules

In a 400 ml beaker were dissolved with stirring (stirring speed: about 1500 rpm) 5.5 g resorcinol in 70 g water and then 2.0 g sodium carbonate solution (20 wt % conc.) were added, whereupon the pH was ca. 7.9. This solution was heated to a temperature of about 52° C. 25.5 g of glutaraldehyde was then added.

The mixture was stirred at a temperature of about 52° C. for a further ca. 10 minutes at a stirring speed of about 1500 rpm (pre-condensation time). About 20 g of water were then added and ca. 2 minutes later 1 g of the protective colloid a) copolymer I.1a, and again ca. 2 minutes later 55 g butylphenyl acetate (CAS-Number 122-43-0; fragrance with a honey-like odor) were added. Immediately afterwards the stirring speed was increased to about 4000 rpm and at approximately the same time 20.0 g sodium carbonate solution (20 wt % conc.) was added The pH of the mixture was then about 9.7. The viscosity and the volume of the mixture then increased. Stirring was continued at about 4000 rpm until the viscosity again dropped. The stirring speed was then lowered to about 1500 rpm. The batch was then stirred at a temperature of about 52° C. at about the same speed for a further 60 minutes. This phase is called the quiescent phase. At the end of this phase the mixture was heated to ca. 80° C. and the capsules were cured at this temperature for a period of 3 hours.

Capsule size distribution—D (90) 5-10 μm; encapsulation efficiency ca. 90%;
Dry yield >90%; solids in the slurry ca. 40 wt %.

In addition to the butylphenyl acetate-containing resorcinol microcapsules of the example 1.2, additional microcapsules were manufactured according to analogous processes:
Example I.3.: Hydroxycitronellal-containing resorcinol microcapsules,
Example I.4.: Helional-containing resorcinol microcapsules,
Example I.5.: Citral-containing resorcinol microcapsules,
Example I.6.: Bourgeonal-containing resorcinol microcapsules,
Example I.7.: Triplal-containing resorcinol microcapsules,
Example I.8.: Ligustral-containing resorcinol microcapsules,
Example I.9.: Vertocitral-containing resorcinol microcapsules,
Example I.10.: Florhydral-containing resorcinol microcapsules,
Example I.11.: Citronellal-containing resorcinol microcapsules,
Example I.12.: Citronellyloxyacetaldehyde-containing resorcinol microcapsules,
Example I.13.: Aloe vera extract-containing resorcinol microcapsules.

Phloroglucinol microcapsules were produced in a further series of examples. In analogy to the process of Example I.2., 6.3 g of pholoroglucinol totally replaced the 5.5 g of resorcinol. Consequently, this resulted in:
Example 1.14.: Butylphenyl acetate-containing phloroglucinol microcapsules,
Example 1.15.: Hydroxycitronellal-containing phloroglucinol microcapsules,
Example 1.16.: Helional-containing phloroglucinol microcapsules,
Example 1.17.: Citral-containing phloroglucinol microcapsules,
Example I.18.: Bourgeonal-containing phloroglucinol microcapsules,
Example I.19.: Triplal-containing phloroglucinol microcapsules,
Example I.20.: Ligustral-containing phloroglucinol microcapsules,
Example I.21.: Vertocitral-containing phloroglucinol microcapsules,
Example I.22.: Florhydral-containing phloroglucinol microcapsules,
Example I.23.: Citronellal-containing phloroglucinol microcapsules,
Example I.24.: Citronellyloxyacetaldehyde-containing phloroglucinol microcapsules,
Example I.25.: Aloe vera extract-containing phloroglucinol microcapsules.

Moreover, in both of the series of examples I.3 to I.13 (resorcinol) or I.14 to I.25 (phloroglucinol) for the synthesis of the microcapsules, 21.9 g of succindialdehyde was used instead of 25.5 g of glutaraldehyde.

### II. Capsule Mixtures

Mixtures of resorcinol microcapsules, obtained by reacting resorcinol with glutardialdehyde according to Example I.2, with phloroglucinol microcapsules, obtained by reacting phloroglucinol with glutardialdehyde according to Example I.2, were produced. All the added microcapsules contained the same perfume composition. The capsule mixtures containing different fractions of resorcinol microcapsules and phloroglucinol microcapsules were obtained by blending the corresponding fractions of each microcapsule (compositions B, C and D). For comparative experiments the relevant capsules were used alone (compositions A and E).

Capsule composition A: 100% perfume-containing phloroglucinol microcapsules

Capsule composition B: 5% perfume-containing phloroglucinol microcapsules and 95% perfume-containing resorcinol microcapsules

Capsule composition C: 10% perfume-containing phloroglucinol microcapsules and 90% perfume-containing resorcinol microcapsules

Capsule composition D: 20% perfume-containing phloroglucinol microcapsules and 80% perfume-containing resorcinol microcapsules

Capsule composition E: 100% perfume-containing resorcinol microcapsules

### III. Examples of Use

All quantitative data refer to wt % active substance, unless otherwise stated.

### Example III.1

### Liquid Conditioner

wt%

| | |
|---|---|
| Esterquat^{[a]} | 15 |
| 2-propanol | 1.7 |
| Silicone oil | 5 |
| MgCl × 6H₂O | 0.5 |
| Microcapsules^{[b]} | 0.2 |
| Water, deionized | ad 100 |

^{[a]}N-Methyl-N(2-hydroxyethyl)-N,N-(ditallowacyloxyethyl)ammonium-methosulfate
^{[b]}Microcapsule-composition according to Example II. A to E

The formulation was produced by melting the esterquat in water. The molten esterquat was then stirred with a high dispersion device and the remaining components were added. After the mixture was cooled down to below 30° C., the perfume and the microcapsules were added with light stirring.

### Example III.2

### Conditioner Substrate

In order to produce the conditioner substrate, cellulose non-wovens (surface area: 24.5×39 cm) were impregnated with 20 g of the liquid conditioner of Example III.1.

### Example III.3

### Liquid Cleaning Agent

| Raw material | Amount in wt % |
|---|---|
| C12-C18 fatty acid, Na salt | 0.7 |
| C10-13 alkylbenzene sulfonate, Na salt | 6.4 |
| Sodium citrate | 1.5 |
| Ethanol | 2.1 |
| Cumene sulfonate, Na | 1.5 |
| C12-18 fatty alcohol + 7EO | 1.5 |
| C8 fatty alcohol sulfate, Na salt | 1.5 |
| Microcapsules^{[a]} | 0.5 |
| Perfume | 0.7 |
| Water | ad 100 |

^{[a]}Microcapsule-composition according to Example II. A to E.

### Example III.5

### Liquid Washing Agent

| Raw material | Amount in wt % |
|---|---|
| C12-14 fatty acid | 8.8 |
| C12-18 fatty alcohol + 7EO | 24.0 |
| Alkyl polyglucoside | 2.0 |
| C12-14-2EO sulfate | 5.0 |
| C16-18 fatty acid | 6.8 |
| NaOH 50% | 3.0 |
| Citric acid × 1H₂O | 1.0 |
| Glycerin 99.5% | 7.5 |
| Ethanol | 1.0 |
| Silicone oil | 0.3 |
| Polyvinyl pyrrolidone | 0.5 |
| HEDP-4Na | 0.5 |
| Enzyme, colorant, perfume | 0.8 |
| Microcapsules^{[a]} | 0.7 |
| Water | ad 100 |

^{[a]}Microcapsule-composition according to Example II. A to E.

### Example III.6

### Solid Washing Agent

| Raw material | Amount in wt % |
|---|---|
| Alkylbenzene sulfonate (sodium salt) | 12 |
| Carboxymethyl cellulose | 1 |
| Enzymes | 1 |
| Non-ionic surfactant | 3 |
| (1-Hydroxyethylidene)bisphosphonate | 1 |
| Sodium carbonate | 25 |
| Sodium percarbonate | 12 |
| Sodium sulfate | 27 |
| Polyacrylate^{[a]} | 3 |
| Defoamer | 2 |
| N,N,N′,N′-tetraacetylethylenediamine | 3 |
| Water | 3 |
| Perfume | 0.15 |
| Microcapsules^{[b]} | 1.0 |
| Sodium silicate^{[c]} | ad 100 |
| Sum total | 100 |

^{[a]}Polyacrylate: polyacrylic acid, sodium salt; M = 4500 g/mol
^{[b]}Microcapsule-composition according to Example II. A to E.
^{[c]}Sodium silicate: amorphous sodium silicate with Na₂O:SiO₂ = 2.4

### Example III.7

### Washing Agent Gel

| Raw material | Amount in wt % |
|---|---|
| Alkyl polyglucoside | 2.00 |
| C12-14 fatty acid | 8.80 |
| C16-18 fatty acid | 6.80 |
| NaOH 50% | 3.00 |
| Citric acid × 1H₂O | 1.00 |
| Glycerin 99.5% | 7.50 |
| Ethanol | 1.00 |
| Silicone defoamer | 0.30 |
| Boric acid | 1.00 |
| 1-Hydroxyethylenediphosphonic acid | 0.50 |
| Vinylimidazole-vinyl pyrrolidone copolymer | 1.67 |
| Microcapsules^{[a]} | 0.8 |
| Perfume | 1.3 |
| Water | ad 100 |

^{[a]}Microcapsule-composition according to Example II. A to E

### Example III.8

### Ironing Spray

| Raw material | Amount in wt % |
|---|---|
| Ethanol | 2 |
| Hydrogen peroxide | 0.01 |
| Perfume | 0.05 |
| Microcapsules^{[c]} | 0.02 |
| Water with 5° dH | ad 100 wt % |

^{[c]}Microcapsule-composition according to Example II. A to E.

### Example III.9

### Antiperspirant-Wax Stick

| Raw material | Amount in wt % |
|---|---|
| PPG-14 Butyl ether | 15.00 |
| Hydrogenated castor oil | 1.00 |
| Stearyl alcohol | 20.00 |
| Ceteareth-30 | 3.00 |
| Perfume | 1.00 |
| Aluminum chlorohydrate | 20.00 |
| Allantoin | 0.10 |
| Cocoglycerides (FP 30-32° C.) | 4.00 |
| Talc | 3.00 |
| Microcapsules^{[a]} | 0.20 |
| Tocopheryl acetate | 0.20 |
| Cyclopentasiloxane | ad 100 |

^{[a]}Microcapsule-composition according to Example II. A to E

### Example III.10

### Suspensions for Spraying as an Antiperspirant Spray

| Raw material | Amount in wt % |
|---|---|
| Perfume | 5.00 |
| Aluminum chlorohydrate (activated) | 30.00 |
| Microcapsules^{[a]} | 2.00 |
| Isopropyl palmitate | 5.00 |
| C12-15 alkyl benzoate | — |
| Cosmacol PLG^{[b]} | — |
| Disteardimonium Hectorite | 4.50 |
| Propylene carbonate | 1.50 |
| Ethylhexyl palmitate | ad 100 |

^{[a]}Microcapsule-composition according to Example II. A to E
^{[b]}Cosmacol PLG (INCI: Di-C12-13 Alkyl tartrate, tri-C12-13 Alkyl Citrat, Silica)

### Example IV

### Washing Tests

For the washing tests, various capsule mixtures from Example II. A to E were tested.

Cotton terrycloth (30×30 cm) was used as the test fabric. The test fabrics were washed with an additional 3 kg of cotton terrycloth and cotton linen union in a washing machine (Miele Softtronic W1734) at 40° C. with 60 ml of unperfumed all-purpose washing powder in the main wash cycle at a water hardness of 16° dH and a spin cycle of 1200 rpm.

The subsequent rinse cycle was carried out with 40 ml of the fabric softener of Example III.1 that comprised a total of 0.20 wt % of the relevant microcapsules of Example II. The test fabrics were hung out to dry at a temperature of 20° C. and a relative humidity of 60% rh.

The boost effects were evaluated after rubbing the dried washing together as well as the coloration and the sedimentation or agglomerate formation in the finished agent of the fresh, after 4 weeks storage at 5° C., after 4 weeks storage at room temperature (RT) and after 4 weeks storage at 40° C., respectively. The evaluation was carried out by qualified perfumers and was repeated two times. The results are shown below.

Results of the Washing Tests

### Example IV.1

### Boost Effect

1=no boost, 2=slight boost, 3=average boost, 4=strong boost, 5=very strong boost

| | | Boost effect | Boost effect | Boost effect |
|---|---|---|---|---|
| Capsule | Boost effect | after rubbing | after rubbing | after rubbing |
| composition | after rubbing | (4 weeks | (4 weeks | (4 weeks at |
| (Example II) | (fresh) | at 5° C.) | at RT) | 40° C.) |
| A | 5 | 5 | 5 | 4 |
| B | 5 | 5 | 4 | 2 |
| C | 5 | 5 | 4 | 3 |
| D | 5 | 5 | 4 | 3.5 |
| E | 5 | 4.5 | 3.5 | 1.1 |

### Example IV.2

### Coloration of the Agent

0=no change in coloration, 1=acceptable, slight change in coloration, 2=average change in coloration, 3=significant change in coloration

| Capsule | | Color | Color | Color |
|---|---|---|---|---|
| composition | Color | (4 weeks | (4 weeks | (4 weeks |
| (Example II) | (fresh) | at 5° C.) | at RT) | at 40° C.) |
| A | 2 | 2 | 2.5 | 2.5 |
| B | 0.3 | 0.3 | 0.7 | 0.7 |
| C | 0.5 | 0.5 | 0.9 | 0.9 |
| D | 0.8 | 0.8 | 1 | 1 |
| E | 0.2 | 0.2 | 0.5 | 0.5 |

### Example IV.3

### Sedimentation or Agglomeration in the Product

0=no sedimentation, 1=acceptable, very slight sedimentation, 2 slight sedimentation, 3=significant sedimentation

| Capsule | Sedimen- | Sedimentation | Sedimentation | Sedimentation |
|---|---|---|---|---|
| composition | tation | (4 weeks at | (4 weeks at | (4 weeks at |
| (Example II) | (fresh) | 5° C.) | RT) | 40° C.) |
| A | 2 | 3 | 3 | 3 |
| B | 1 | 1.5 | 1.5 | 1.5 |
| C | 1 | 1.7 | 1.7 | 1.7 |
| D | 1 | 2 | 2 | 2 |
| E | 0.5 | 1.2 | 1.2 | 1.2 |

As is shown by the tests, a significant sedimentation occurs with the pure phloroglucinol microcapsules (capsule composition A) during storage. Moreover, these capsules afford average to significant changes in coloration of the finished agent for washing, cleaning, conditioning, caring and/or dyeing hard or soft surfaces. The advantage of the phloroglucinol microcapsules, however, is their strong to very strong boost effect also even after four weeks. In contrast, with pure resorcinol microcapsules (capsule composition E), no change in coloration and only very slight sedimentation occurs. The boost effect, however, decreases very strongly during storage, in particular after four weeks at 40° C. the boost effect is no longer present.

The tests clearly show that only a slight color change of the end product with slight sedimentation of the capsules occurs with a combination of phloroglucinol microcapsules and resorcinol microcapsules (capsule compositions B, C and D). However, the boost effect surprisingly increases to a significantly higher degree than would be expected from the amounts of phloroglucinol microcapsules. In particular, after storage at 40° C., the synergistic effect of the capsule compositions B, C and D is particularly significant when compared to the pure capsule compositions A and E.

Similarly good results for the capsule compositions B, C and D were achieved with other washing, cleaning or post-treatment agents (Examples III.2 to III.8) as well as with cosmetic compositions (Examples III.9 and III.10).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for releasing an active substance from a microcapsule that is present on a surface, wherein the microcapsule, through contact with an agent for washing, cleaning, conditioning, and/or dyeing hard or soft surfaces, comprising
i. at least one surfactant, and
ii. a mixture of at least a first and a second microcapsule, which each comprise at least one active substance and whose capsule walls comprise a resin that is obtained by reacting
a) at least one aromatic alcohol or its ethers or derivatives with
b) at least one aldehydic component that possesses at least two carbon atoms per molecule, and
c) optionally in the presence of at least one (meth)acrylate polymer,
wherein the first and the second microcapsules differ from one another in one of the reacted components a) and/or b),
is deposited onto the surface and then releases the active substance by the application of mechanical force.

2. The method according to claim 1, wherein the active substance is released by friction.

3. The method according to claim 1, wherein the surface concerns a textile surface and the agent is a washing, cleaning or post-treatment agent.

4. The method according to claim 1, wherein the first and the second microcapsules differ from one another in the reacted components a).

5. The method according to claim 1, wherein the first and the second microcapsules differ from one another in the reacted components b).

6. The method according to claim 1, wherein the aromatic alcohol a) is selected from the group consisting of phenols, o-cresol, m-cresol, p-cresol, α-naphthol, β-naphthol, thymol, pyrocatechol, resorcinol, hydroquinone, 1,4-naphthohydroquinone, phloroglucinol, pyrogallol, hydroxyhydroquinone and mixtures thereof.

7. The method according to claim 1, wherein the aldehydic component b) is selected from the group consisting of valeraldehyde, capronaldehyde, caprylaldehyde, decanal, succindialdehyde, cyclohexane carbaldehyde, cyclopentane carbaldehyde, 2-methyl-1-propanal, 2-methylpropionaldehyde, acetaldehyde, acrolein, aldosterone, antimycin A, 8′-apo-β-caroten-8′-al, benzaldehyde, butanal, chloral, citral, citronellal, crotonaldehyde, dimethylaminobenzaldehyde, folic acid, fosmidomycin, furfural, glutaraldehyde, glycerin aldehyde, glycolaldehyde, glyoxal, glyoxylic acid, heptanal, 2-hydroxybenzaldehyde, 3-hydroxybutanal, hydroxymethylfurfural, 4-hydroxynonenal, isobutanal, isobutyraldehyde, methacrolein, 2-methylundecanal, mucochloric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicyl aldehyde, secologanin, streptomycin, strophanthidin, tylosin, vanillin, cinnamaldehyde and mixtures thereof.

8. The method according to claim 1, wherein the first microcapsule comprises phloroglucinol and the second microcapsule comprises resorcinol as the aromatic alcohol a).

9. The method according to claim 8, wherein it comprises the first and the second microcapsule in a ratio of 1:30 to 1:3.

10. The method according to claim 1, wherein the microcapsules comprise 0.0001 to 50 wt %, in each case based on the total agent.
